(19) United States
(12) Patent Application Publication
(10) Pub. No.: US 2009/0130153 A1
(43) Pub. Date: May 21, 2009

(54) Emulsifier Combination, Emulsion Containing the Emulsifier Combination, and a Process for its Production

(75) Inventors: Ulrich Issberner, Rommerskirchen (DE); Caroline Goget, Dusseldorf (DE)

Correspondence Address: FOX ROTHSCHILD LLP, 2000 MARKET STREET, PHILADELPHIA, PA, 19103, US

(73) Assignee: Cognis IP Management GmbH, Duesseldorf (DE)

(21) Appl. No.: 12/359,791

(22) Filed: Jan. 26, 2009

Related U.S. Application Data
(62) Division of application No. 10/899,670, filed on Jul. 27, 2004.

(30) Foreign Application Priority Data
Jul. 28, 2003 (DE) 103 34 225.7
Oct. 2, 2003 (DE) 103 46 515.4

(51) Int. Cl. A61K 8/60

(52) U.S. Cl. 424/401; 977/915; 977/926

(57) ABSTRACT

An emulsifier concentrate containing: (a) a C₈₋₁₄ alkyl oligoglycoside, (b) a polyol hydroxystearate; (c) a C₆₋₂₂ acyl glutamate; (d) water; and (e) optionally, at least one auxiliary component, and wherein the composition is ethoxylate-free.

### CROSS REFERENCE TO RELATED APPLICATIONS

This application is a division of U.S. application Ser. No. 10/899,670, filed Jul. 27, 2004 which claims priority from German patent application number DE 103 34 225.7, filed Jul. 28, 2003 and German patent application number DE 103 46 515.4, filed Oct. 2, 2003, which are incorporated herein by reference in their entireties.

### BACKGROUND OF THE INVENTION

This invention relates to an emulsifier combination which, although free from ethoxylated surfactants, gives stable nanoemulsions in conventional cold/cold or hot/hot emulsification processes. The present invention also relates to the nanoemulsions produced using the emulsifier combination according to the invention and to a process for their production.

Nanoemulsions are normally understood to be emulsions having a particle or droplet size below 1,000 nm. More particularly, nanoemulsions are emulsions with a mean particle size in the range from ca. 5 to 500 nm.

By virtue of their favorable properties, nanoemulsions are often used in cosmetic and pharmaceutical preparations. Their advantages include, above all, their phase stability, even at low viscosities, and their distinctly higher absorption capacity for active components applied with the emulsion, for example to the skin or hair, by comparison with conventional emulsions.

Hitherto, stable nanoemulsions have been produced almost exclusively by the phase inversion temperature (PIT) process. However, only ethoxylated emulsifiers may be used in this process. Emulsifiers such as these often irritate the skin and are therefore a disadvantage. Emulsions obtained by the PIT process are described, for example, in Applicants' DE 19541754 A1.

An alternative to the PIT process, where the ethoxylated emulsifiers may be at least partly replaced by non-ethoxylated emulsifiers, is disclosed in applicants' DE 10059430 A1. Here, the aqueous and oil phases are emulsified by high-pressure homogenization. The production process is therefore relatively complicated and expensive.

Accordingly, there was a need for an emulsifier or an emulsifier combination where there would be no need to use ethoxylated emulsifiers in order both simply and inexpensively to obtain stable nanoemulsions. Accordingly, the problem addressed by the present invention was to provide such an emulsifier combination and also products coated with ethoxylate-free nanoemulsions.

### DESCRIPTION OF THE INVENTION

The problem stated above has been solved by the emulsifier combination claimed in claim 1. Preferred embodiments are described in the subsidiary claims. Accordingly, in a first embodiment, the present invention relates to an emulsifier combination which is free from ethoxylated nonionic emulsifiers, contains less than 20% by weight water and comprises a) C₈₋₁₄ alkyl oligoglycoside, b) polyol polyhydroxystearate and c) C₆₋₂₂ acyl glutamate.

In the context of the invention, the term “emulsifier” is understood to encompass both emulsifiers and surfactants, i.e. surface-active substances in general, and is used representatively in the following for all such compounds.

### Alkyl Oligoglycoside

Alkyl oligoglycosides are known nonionic surfactants which correspond to formula (I):

R¹O-[G]ₚ (I)

where R¹ is an alkyl group, G is a sugar unit containing 5 or 6 carbon atoms and p is a number of 1 to 10. They may be obtained by the relevant methods of preparative organic chemistry. EP 0 301 298 A1 and WO 90/03977 A are cited as representative of the extensive literature available on this subject.

The alkyl oligoglycosides may be derived from aldoses or ketoses containing 5 or 6 carbon atoms, preferably glucose. Accordingly, the preferred alkyl oligoglycosides are alkyl oligoglucosides.

The index p in general formula (I) indicates the degree of oligomerization (DP degree), i.e. the distribution of mono- and oligoglycosides, and is a number of 1 to 10. Whereas p in a given compound must always be an integer and, above all, may assume a value of 1 to 6, the value p for a certain alkyl oligoglycoside is an analytically determined calculated quantity which is generally a broken number. Alkyl oligoglycosides having an average degree of oligomerization p of 1.1 to 3.0 are preferably used. Alkyl oligoglycosides having a degree of oligomerization of less than 1.7 and, more particularly, between 1.2 and 1.4 are preferred from the applicational perspective.

According to the invention, it has been found that the length of the alkyl chain R¹ appears to be a particular factor in the suitability of the emulsifier combination according to the invention for the production of nanoemulsions. Accordingly, alkyl oligoglycosides where R¹ is derived from primary C₈₋₁₄ alcohols are used for the purposes of the invention. Technical mixtures of the alcohols may also be used. The C₈₋₁₂ alkyl groups are preferred. As already mentioned, the corresponding alkyl oligoglycosides—especially lauryl glucoside—are particularly suitable.

### Polyol Polyhydroxystearates

Polyol polyhydroxystearates are esters of polyols and polyhydroxystearic acids. The polyol component may be derived, for example, from glycerol, ethylene glycol, diethylene glycol, propylene glycol, polyglycerol, trimethylol ethane, trimethylol propane, trimethylol butane, pentaerythritol, dipentaerythritol, methyl and butyl glucoside, sorbitol, mannitol, glucose, sucrose or glucamine. Corresponding substances are known, for example, from GB-A 1524782 or EP-A-0000424.

The substances of component b) are preferably polyglycerol polyhydroxy stearates which are obtained by esterifying polyhydroxystearic acid with a degree of self-condensation of 2 to 20 and preferably 2 to 10 with a polyglycerol mixture having the following preferred composition (gas chromatography):

| | | |
|---|---|---|
| glycerol | 5 to 35 (15 to 30) % | by weight |
| diglycerols | 15 to 40 (20 to 32) % | by weight |
| triglycerols | 10 to 35 (15 to 25) % | by weight |
| tetraglycerols | 5 to 20 (8 to 15) % | by weight |
| pentaglycerols | 2 to 10 (3 to 8) % | by weight |
| oligoglycerols | to 100% | by weight |

by methods known per se. The preferred ranges are shown in brackets. The polyol polyhydroxystearates may be produced by methods known per se. In the case of the polyglycerol polyhydroxystearates, the polyglycerol and the polyhydroxystearic acid are preferably produced in that order and, finally, both are esterified. A polyglycerol with the composition indicated above may be prepared by self-condensation of glycerol in the presence of suitable catalysts, for example potassium carbonate, silicates according to DE 4029323 (Henkel) or borates according to DE 4117033 (Henkel) at temperatures of 200 to 260° C. The polyhydroxystearic acid is produced, for example, by alkali-catalyzed polycondensation of hydroxystearic acid, preferably 12-hydroxystearic acid, which is obtained by hydrogenation of ricinoleic acid or technical castor oil fatty acid. Linear esterification products containing 2 to 10 and, more particularly, 2 to 8 fatty acid units are preferably formed. The following distribution (GPC method) is typically achieved:

| | | |
|---|---|---|
| monomers | 1 to 10% | by weight |
| dimers | 5 to 15% | by weight |
| trimers | 5 to 15% | by weight |
| tetramers | 5 to 15% | by weight |
| pentamers | 5 to 15% | by weight |
| hexamers | 5 to 15% | by weight |
| heptamers | 5 to 15% | by weight |
| octamers | 1 to 10% | by weight |
| oligomers | to 100% | by weight |

One particular embodiment of the invention is characterized by the use of mixtures of hydroxystearic acid and ricinoleic acid or technical castor oil fatty acid, of which around 90% by weight consists of ricinoleic acid, in a ratio by weight of 99:1 to 1:99 and preferably 75:25 to 10:90. Similarly, the acids may be individually condensed and the resulting condensates mixed. A complex mixture of homologous polyesters is formed in the subsequent condensation of the polyol component, for example the polyglycerol, with the polyhydroxystearic acid or the mixtures with polyricinoleic acid. Condensation products based on polyglycerol and polyhydroxystearic acid or polyhydroxystearic acid/polyricinoleic acid may be characterized by their iodine value. Typical examples are polyesters with an iodine value below 10 (basis 100% 12-hydroxystearic acid) or 65 to 80 (basis 90% 12-hydroxystearic acid, 10% ricinoleic acid).

### Acyl Glutamates

Acyl glutamates are known anionic surfactants corresponding to formula (II):

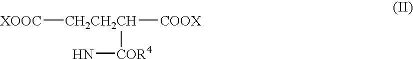

in which R⁴CO is a linear or branched acyl group containing 6 to 22 carbon atoms and 0, 1, 2 or 3 double bonds and X is hydrogen, an alkali metal and/or alkaline earth metal, ammonium, alkylammonium, alkanolammonium or glucammonium. They are produced, for example, by Schotten-Baumann acylation of glutamic acid with fatty acids, fatty acid esters or chlorides. Corresponding commercial products are available, for example, from Hoechst AG, Frankfurt, FRG or from the Ajinomoto Co. Inc., Tokyo, JP. An overview of the production and properties of acyl glutamates was published by M. Takehara et al. in J. Am. Oil. Chem. Soc., 49, (1972) 143. Typical examples of acyl glutamates suitable for the purposes of the invention are those derived from fatty acids containing 6 to 22 and preferably 12 to 18 carbon atoms. Coconut oil fatty acid glutamates, for example of C_{12/14} or C_{12/18} coconut oil fatty acid, are particularly preferred. The mono- or dialkali metal salts of acyl glutamate in particular are used.

The emulsifier concentrates are also suitable for solubilizing perfume oils. Accordingly, the present invention also relates to the use of the emulsifier concentrates for solubilizing perfume oils.

### Other Emulsifiers

In addition to components a) to c) mentioned above, the emulsifier combination according to the invention may contain other emulsifiers or surfactants compatible with components a) to c). The choice is governed by the intended use of the emulsifier combination and, in particular, by the nature of the oil component to be emulsified. According to the invention, ethoxylated emulsifiers are not included among the other emulsifiers. At least one other nonionic surfactant is preferably present as an additional component. Sorbitan monoesters or diesters of a saturated or unsaturated fatty acid containing 6 to 22 and preferably 12 to 20 carbon atoms, especially sorbitan stearate, are particularly suitable.

The quantities in which components a) to c) and optionally the other emulsifiers are used in the emulsifier combination according to the invention are also determined by the composition of the oil phase to be emulsified. Component c) preferably makes up 1 to 50% by weight, more preferably 3 to 15% by weight and most preferably 5 to 11% by weight of the emulsifier combination.

The ratio by weight of component a) to component b) is in the range from 5:1 to 1:5 and preferably in the range from 2:1 to 1:2. In a particularly favorable embodiment, the two components are present in substantially equal quantities by weight.

If another surfactant/another emulsifier is present in the emulsifier combination according to the invention, its ratio by weight to components a) and b) together is in the range from 1:1 to 1:10 and preferably in the range from 1:2 to 1:5.

The emulsifier combination according to the invention may be used in the form of a concentrate of all the above-mentioned emulsifier components with a percentage water content of less than 20% by weight water. The percentage water content of the emulsifier combination according to the invention is preferably less than 10% by weight and more particularly less than 5% by weight.

Glycerol is preferably added to the emulsifier combination, leading in combination with the water to a liquid and pumpable emulsifier composition.

However, the emulsifier combination according to the invention may also be used in the form of its individual components or in the form of mixtures of the individual components, in which case the percentage water content of the individual components as a whole or mixtures thereof is under 20% by weight. Accordingly, in the production of the emulsion, not only can the emulsifier combination according to the invention as a whole be initially introduced with the oil phase, the components of the emulsifier combination may also be divided between the oil phase and the aqueous phase, preferably in such a way that the oil-soluble components are added to the oil phase and the water-soluble components to the aqueous phase.

### Nanoemulsion

Basically, the emulsifier combination according to the invention is suitable for the production of nanoemulsions of any oil components/emollients typically used in cosmetic or pharmaceutical emulsions. Accordingly, the present invention also relates to nanoemulsions in which an oil phase and an aqueous phase are emulsified with one another, in which the described emulsifier combination is present as emulsifier and which are free from ethoxylated emulsifiers. In the context of the invention, nanoemulsions are emulsions with a particle or droplet size below 1,000 nm. The particle size of the nanoemulsion according to the invention is typically in the range from 5 to 500 nm and more particularly in the range from 50 to 200 nm.

Suitable oil components are, for example, Guerbet alcohols based on fatty alcohols containing 6 to 18 and preferably 8 to 10 carbon atoms (for example Eutanol® G), esters of linear C₆₋₂₂ fatty acids with linear or branched C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₃₋₃₈ alkylhydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, such as 1,3-dialkyl cyclohexanes for example, linear and branched C₆₋₂₂ fatty alcohol carbonates, such as Dicaprylyl Carbonate (Cetiol® CC) for example, Guerbet carbonates based on C₆₋₁₈ and preferably C₈₋₁₀ fatty alcohols, esters of benzoic acid with linear and/or branched C₆₋₂₂ alcohols (for example FinsolvφTN), linear or branched, symmetrical or nonsymmetrical dialkyl ethers containing 6 to 22 carbon atoms per alkyl group, such as Dicaprylyl Ether (Cetiol® OE) for example, ring opening products of epoxidized fatty acid esters with polyols (Hydagen® HSP, Sovermol® 750, Sovermol® 1102), silicone oils (cyclomethicone, silicon methicone types, etc.) and/or aliphatic or naphthenic hydrocarbons such as, for example, mineral oil, Vaseline, petrolatum, squalane, squalene or dialkyl cyclohexanes.

Preferred oil components in the nanoemulsion according to the invention are selected from fatty acid esters of C₆₋₂₂ fatty acids with C₆₋₂₂ fatty alcohols, C₆₋₂₂ fatty alcohol carbonates, silicone oils, 1,3-dialkyl cyclohexanes and C₆₋₂₂ dialkyl ethers.

Particularly good results are obtained when the nanoemulsion contains the following components:
a) C₈₋₁₂ alkyl oligoglycoside, more particularly lauryl glucoside,
b) polyglycerol polyhydroxystearate,
c) C₁₂₋₁₈ acyl glutamate, more particularly coconut oil fatty acid glutamate,
d) sorbitan esters of a C₁₆₋₂₀ fatty acid, more particularly sorbitan stearate,
   at least one oil component and preferably at least one oil component per group selected from
e) fatty acid esters of C₆₋₂₀ fatty acids with C₆₋₂₀ fatty alcohols, more particularly hexyl laurate, ethyl hexyl palmitate and cetearyl isononanoate,
f) C₆₋₁₂ dialkyl carbonates, more particularly dicaprylyl carbonate, and
g) cyclomethicones and silicon methicones, more particularly dimethicones, and
h) glycerol.
i) water and optionally
j) active components, auxiliaries and additives.

Instead of components a), b) and i), an emulsifier composition marketed under the name of Eumulgin® VL 75 by Cognis Deutschland GmbH & Co. KG, which contains lauryl glucoside, polyglyceryl-2-dipolyhydroxystearate and glycerol may also be used with advantage.

In a preferred embodiment, the nanoemulsion additionally contains C₁₆₋₂₂ fatty alcohol(s), more particularly cetyl and cetyl stearyl alcohol, in a quantity of preferably 1.0 to 10% by weight.

Active components in the context of the invention include inter alia medicaments, cosmetic or pharmaceutical active components such as, for example, antioxidants, UV filters, biogenic agents, deodorants, self-tanning agents, depigmenting agents, perfume oils or other perfumes, etc. Hydrotropes, fillers, swelling agents, stabilizers (for example polysaccharide, acrylic acid, acrylamide polymers), preservatives, dyes and the other usual auxiliaries and additives, which are normally used in only small quantities, may also be present.

The quantity in which the emulsifier combination according to the invention is used for emulsification is determined in known manner by the nature and composition of the oil component to be emulsified. The percentage content of component c) is preferably 0.05 to 3% by weight and more particularly 0.1 to 1.5% by weight, based on the total quantity of the nanoemulsion. The percentage content of additional emulsifier components is generally 0.1 to 15% by weight and more particularly 1 to 10% by weight, again based on the total quantity of nanoemulsion. Oil component (emollient) is present in the nanoemulsion in a quantity of preferably 1 to 50% by weight and more particularly 1 to 30% by weight, based on the total quantity of nanoemulsion.

The percentage water content is generally above 20% by weight, preferably above 40% by weight and more particularly above 50% by weight.

A most particularly preferred embodiment of the nanoemulsion comprises:
a) 0.5 to 5.0% by weight C₈₋₁₂ alkyl oligoglycoside, more particularly lauryl glucoside,
b) 0.5 to 5.0% by weight polyglycerol polyhydroxystearate,
c) 0.5 to 5.0% by weight C₁₂₋₁₈ acyl glutamate, more particularly coconut oil fatty acid glutamate,
d) 0.5 to 5.0% by weight sorbitan ester of a C₁₆₋₂₀ fatty acid, more particularly sorbitan stearate,
e) 5.0 to 20.0% by weight fatty acid esters of C₆₋₂₀ fatty acids with C₆₋₂₀ fatty alcohols, more particularly hexyl laurate, ethyl hexyl palmitate and cetearyl isononanoate,
f) 1.0 to 10.0% by weight C₆₋₁₂ dialkyl carbonates, more particularly dicaprylyl carbonate, and
g) 0.1 to 5.0% by weight cyclomethicones and/or silicon methicones, more particularly dimethicone, and
h) 1.0 to 10% by weight glycerol.
i) up to 90% by weight water and optionally
j) active components, auxiliaries and additives.

Another preferred embodiment is characterized in that the nanoemulsion consists essentially of components a)-j). In the content of the invention, “essentially” means that, apart from impurities attributable to the raw materials, the nanoemulsion contains no components other than components a)-j).

### Production Process

In the production of the emulsion, the emulsifier combination according to the invention as a whole may be initially introduced with the oil phase. At present, however, it is preferred to divide the components of the emulsifier combination between the oil phase and the aqueous phase so that the oil-soluble components are added to the oil phase and the water-soluble components to the aqueous phase.

Accordingly, in the process according to the invention for the production of a nanoemulsion, an oil phase containing the oil component, optionally other oil-soluble components and emulsifier components a) and b) is initially introduced and the aqueous phase which contains emulsifier component c) and optionally other water-soluble components is stirred into and emulsified in the oil phase.

Accordingly, a simple mixing process is sufficient for obtaining a nanoemulsion. There is no need for a PIT process, high-pressure homogenization or any other elaborate process to produce a stable nanoemulsion. In addition, the emulsions may be produced without any ethoxylated compounds whatever.

Basically, both w/o and o/w emulsions may be produced with the emulsifier combination according to the invention. The emulsifier combination and the process according to the invention are particularly suitable for the production of phase-stable o/w emulsions which have a low viscosity (generally below 100 mPas).

So far as introduction of the aqueous phase into the oil phase is concerned, about 5 to 10% by volume of the aqueous phase is initially introduced slowly while stirring. The mixture remains relatively clear. As more aqueous phase is added, the mixture slowly clouds and becomes opalescent blue and reflective. A milky emulsion is ultimately obtained.

It is assumed that, in the emulsification process, the emulsifier components are increasingly solubilized in the oil phase as the water content of the emulsion increases; at the same time, the acyl glutamate synergistically contributes towards increasing the hydrophilia of the mixture, so that an o/w emulsion is able to form in this way.

In order that the acyl glutamate may develop its full effect and to prevent the acyl glutamate being present in the form of glutamic acid, the pH of the aqueous phase is preferably adjusted to a value of at least 4.5. If necessary, the final emulsion may then be adjusted to the desired pH.

In addition, the particle or droplet size of the final emulsion may also be controlled through the pH adjusted in the aqueous phase to be emulsified. In a preferred variant of the process, the pH of the aqueous phase is adjusted in dependence upon the desired particle size in the emulsion obtained. The particle size of the nanoemulsion decreases with increasing pH. This would appear to be attributable to the relatively strong ionization of the acyl glutamate. The chemicals usable for adjusting the pH are known to the expert and, purely by way of example, include citric acid and alkali metal hydroxides.

The process according to the invention is not confined to particular temperature ranges and may be carried out as a hot/hot or cold/cold process. In contrast to the PIT process, phase inversion in the process according to the invention is not temperature-dependent. The suitable temperature depends in known manner on the components used. If viscous, wax-like or solid components are to be emulsified, the process is generally carried out at elevated temperature. For example, the oil phase and the aqueous phase may be emulsified with one another at an elevated temperature of, in particular, 40 to 80° C.

### Commercial Applications

The emulsifier combination according to the invention is suitable for the production of nanoemulsions free from ethoxylated emulsifiers which may be used with advantage in cosmetic or pharmaceutical preparations. Examples include sprayable body-care emulsions (deodorant sprays, sun protection sprays, etc.), care products in gel or cream form, medicament-containing sprays, gels or creams, impregnated wipes or pads (makeup removers, cleaning wipes, etc.) and the like. The nanoemulsions are particularly suitable for application to papers, cloths, textiles and cottonwool products used in baby care and hygiene and in the removal of makeup, particularly eye makeup, in feminine hygiene (tampons, sanitary napkins, panty liners) and in personal hygiene (toilet papers, moist toilet papers) because allergy problems are particularly common in such fields and have to be avoided.

Commercially available cleaning wipes are impregnated either with aqueous lotions based on ethoxylate-containing (EO) emulsifiers, as described in WO 00/04230 for example, or with aqueous clear solubilizates. The aqueous lotions mentioned are not accepted in various markets because of their EO content (“eco-compliance”). By contrast, the clear solubilizates are often very tacky and are unacceptable from the sensory perspective. Since solubilizates often have to be heavily diluted because of their tackiness, the wipes produced with them show poor cleaning performance. The nanoemulsions according to the invention contain small quantities of emulsifiers and larger quantities of oil and wax. Accordingly, their cleaning performance is very good. In addition, they are free from ethoxylate-containing emulsifiers.

Accordingly, the present invention also relates to the use of the nanoemulsion according to the invention on papers, nonwovens and wovens. According to the invention, these include all types of paper, nonwovens and wovens which are known to the expert and products that can be produced from them such as, for example, toilet papers, paper handkerchiefs, tissues, wipes, cottonwool, cotton pads, tampons, sanitary napkins, panty liners, textiles, etc. The present invention also relates to paper, nonwoven and woven products for body care and personal hygiene which are coated with a nanoemulsion according to the invention.

In a particularly advantageous embodiment, the nanoemulsions are used with a high water content so that they can be applied particularly easily in industrial processes, for example by spraying. Corresponding nanoemulsions have a minimum water content of 60% by weight, preferably 70% by weight and more particularly above 80% by weight, based on the overall composition of the emulsion. Accordingly, the present invention relates to paper, nonwoven or woven products coated with a nanoemulsion according to the invention which is characterized in that it comprises the following components:
a) 0.10 to 3.0% by weight C₈₋₁₂ alkyl oligoglucoside, more particularly lauryl glucoside,
b) 0.10 to 3.0% by weight polyglycerol polyhydroxystearate,
c) 0.02 to 3.0% by weight C₁₂₋₁₈ acyl glutamate, more particularly coconut oil fatty acid glutamate,
d) 0.5 to 10.0% by weight of an oil phase,
e) 0.1 to 3% by weight glycerol,
f) more than 60% by weight and preferably more than 80% by weight water and optionally
g) active components, auxiliaries and additives,
   the quantities shown being based on the overall composition of the emulsion.

Another preferred embodiment is characterized in that the nanoemulsion consists essentially of components a)-g). In the content of the invention, “essentially” means that, apart from impurities attributable to the raw materials, the nanoemulsion contains no components other than components a)-g).

The paper, nonwoven or woven products may be aftertreated in a drying step to reduce the water content after application by spraying or to obtain substantially water-free products (for example dry wipes). In one embodiment of the invention, paper, nonwoven and woven products coated in accordance with the invention are subsequently subjected to a drying step to remove all or part of the water.

The following Examples are intended to illustrate the invention.

### EXAMPLES

Unless otherwise indicated, the quantities mentioned in the following Examples represent % by weight of the commercially available substances or, where no commercially available substance is mentioned, the quantity of active substance, based on the composition as a whole (phase 1 and phase 2). The particle or droplet size of the emulsions was determined by PCS (photon correlation spectroscopy) using a Pacific Scientific NICOMP 370 spectrometer.

### Example 1

An oil phase (phase 1) and an aqueous phase (phase 2) with the compositions shown in Table 1 were prepared. The quantities shown are based on the final emulsion, i.e. the sum of the components of phases 1 and 2 comes to 100% by weight. Both phases were heated to 70° C. Phase 2 was slowly added with stirring to the oil phase 1. The mixture obtained initially remained clear until after ca. 5% of the aqueous phase 2 had been added. When more of the aqueous phase was added, the mixture became an opalescent reflective blue in color. After all of phase 2 had been added and the mixture cooled to room temperature, a milky phase-stable emulsion was obtained. The viscosity of the emulsion was below 100 mPas. The average particle size was 160 nm.

TABLE 1

| | | | |
|---|---|---|---|
| Manufacturer | Name | INCI Name | % by wt. |

PHASE 1

| | | | |
|---|---|---|---|
| Cognis Deutschland | Lauryl glucoside | Lauryl Glucoside | 2.28 |
| GmbH &Co. KG | | | |
| Cognis Deutschland | Dehymuls ® PGPH | Dipolyhydroxystearate | 2.28 |
| GmbH &Co. KG | | | |
| | Glycerol | Glycerin | 1.72 |
| Cognis Deutschland | Dehymuls ® SMS | Sorbitan Stearate | 1.38 |
| GmbH &Co. KG | | | |
| Cognis Deutschland | Lanette ® O | Cetearyl Alcohol | 4.30 |
| GmbH &Co. KG | | | |
| Cognis Deutschland | Cegesoft ® C24 | Octyl Palmitate | 13.80 |
| GmbH &Co. KG | | | |
| Cognis Deutschland | Cetiol ® CC | Dicaprylyl Carbonate | 6.90 |
| GmbH &Co. KG | | | |
| Dow Corning | DC Fluid 200-50 cSt | Dimethicone | 0.50 |

PHASE 2

| | | | |
|---|---|---|---|
| Laboratoires | Water | Water | to 100 |
| Sérobiologiques, FR | Elestab ® 50J (0.3%) | Chlorphenesin and | qs |
| | Glycerol | Methylparaben | 6.00 |
| | NaOH 10% | Glycerin | 0.80 |
| Ajinomoto, JP | Acylglutamate ® CS 11 | Sodium Hydroxide | 1.10 |
| | | Sodium Cocoyl Glutamate | |
| | pH: 7.1 | | |

### Example 2

The emulsion was produced as described in Example 1 using phases 1 and 2 shown in Table 2. The viscosity of the emulsion was below 100 mPas. The average particle size was 170 nm.

TABLE 2

| | | | |
|---|---|---|---|
| Manufacturer | Name | INCI Name | % by wt. |

PHASE 1

| | | | |
|---|---|---|---|
| Cognis Deutschland | Lauryl glucoside | Lauryl Glucoside | 1.06 |
| GmbH &Co. KG | | | |
| Cognis Deutschland | Dehymuls ® PGPH | Dipolyhydroxystearate | 1.06 |
| GmbH &Co. KG | | | |
| | Glycerol | Glycerin | 0.80 |
| Cognis Deutschland | Dehymuls ® SMS | Sorbitan Stearate | 0.80 |
| GmbH &Co. KG | | | |
| Cognis Deutschland | Lanette ® 16 | Cetyl Alcohol | 2.00 |
| GmbH &Co. KG | | | |
| Cognis Deutschland | Cetiol ® SN | Cetearyl Isononanoate | 3.00 |
| GmbH &Co. KG | | | |
| Cognis Deutschland | Cetiol ® CC | Dicaprylyl Carbonate | 3.00 |
| GmbH &Co. KG | | | |
| Cognis Deutschland | Cetiol ® A | Hexyl Laurate | 2.00 |
| GmbH &Co. KG | | | |
| Dow Corning | DC Fluid 200-50 cSt | Dimethicone | 0.50 |

PHASE 2

| | | | |
|---|---|---|---|
| Laboratoires | Water | Water | to 100 |
| Sérobiologiques, FR | Elestab ® 50J (0.3%) | Chlorphenesin and | qs |
| | Glycerol | Methylparaben | 3.00 |
| Ajinomoto, JP | Acylglutamate ® CS 11 | Glycerin | 0.30 |
| | | Sodium Cocoyl Glutamate | |
| | pH: 5.6 | | |

### Example 3

The emulsion was produced in the same way as in Example 2 with the only difference that, before mixing with phase 1, the aqueous phase 2 was adjusted to a pH of 8.5 by addition of sodium hydroxide. The resulting nanoemulsion had a particle size of 110 nm. The emulsion obtained was then adjusted to a pH of 6.9 by addition of dilute citric acid.

### Example 4

Water and an active component were added at room temperature to the nanoemulsion obtained in Example 1. The active component Slimfit® LS 9609 (Laboratoires Sérobiologiques, FR) is intended for cosmetic, local weight reduction. A sprayable body lotion was obtained by mixing
50% by weight of the nanoemulsion of Example 1,
48% by weight water and
2% by weight Slimfit® LS 9609.

### Example 5

The emulsion was produced as in Example 1 using phases 1 and 2 of Table 3. The emulsion remained clear. The viscosity of the emulsion was below 100 mPa·s. The average particle size was 25 nm.

TABLE 3

| | | | |
|---|---|---|---|
| Manufacturer | Name | INCI Name | % by wt. |

PHASE 1

| | | | |
|---|---|---|---|
| Cognis Deutschland | Lauryl glucoside | Lauryl Glucoside | 8.00 |
| GmbH &Co. KG | | | |
| Cognis Deutschland | Dehymuls ® PGPH | Dipolyhydroxystearate | 8.00 |
| GmbH &Co. KG | | | |
| Cognis Deutschland | Glycerol | Glycerin | 6.00 |
| GmbH &Co. KG | | | |
| Cognis Deutschland | Myritol ® 331 | Cocoglycerides | 4.00 |
| GmbH &Co. KG | | | |
| Cognis Deutschland | Eutanol ® G | Octyldodecanol | 2.00 |
| GmbH &Co. KG | | | |
| | Cetiol ® CC | Dicaprylyl Carbonate | 4.00 |

PHASE 2

| | | | |
|---|---|---|---|
| Cognis Deutschland | Plantapon ® ACG 35 | Disodium Cocoyl Glutamate | 0.35 |
| GmbH &Co. KG | Preservative | | q.s. |
| | Citric Acid | Citric Acid | q.s. |
| | Water | Water | to 100 |

pH 5-6

### Example 6

The emulsion was produced as in Example 1 using phases 1 and 2 of Table 4. A phase-stable, clear, bluish emulsion was obtained. The viscosity of the emulsion was below 100 mPa·s. The average particle size was 32 nm.

TABLE 4

| | | | |
|---|---|---|---|
| Manufacturer | Name | INCI Name | % by wt. |

PHASE 1

| | | | |
|---|---|---|---|
| Cognis Deutschland | Lauryl glucoside | Lauryl Glucoside | 8.00 |
| GmbH &Co. KG | | | |
| Cognis Deutschland | Dehymuls ® PGPH | Dipolyhydroxystearate | 8.00 |
| GmbH &Co. KG | | | |
| Cognis Deutschland | Glycerol | Glycerin | 6.00 |
| GmbH &Co. KG | | | |
| Cognis Deutschland | Myritol ® 331 | Cocoglycerides | 4.00 |
| GmbH &Co. KG | | | |
| Cognis Deutschland | Eutanol ® G | Octyldodecanol | 2.00 |
| GmbH &Co. KG | | | |
| Cognis Deutschland | Cetiol ® CC | Dicaprylyl Carbonate | 4.00 |
| GmbH &Co. KG | | | |
| Cognis Deutschland | Lanette ® O | Cetearyl Alcohol | 2.00 |
| GmbH &Co. KG | | | |

PHASE 2

| | | | |
|---|---|---|---|
| Cognis Deutschland | Plantapon ® ACG 35 | Disodium Cocoyl Glutamate | 0.35 |
| GmbH &Co. KG | Preservative | Preservative | q.s. |
| | Citric Acid | Citric Acid | q.s. |
| | Water | Water | to 100 |

pH 5-6.5

### Example 7

The emulsion was produced as in Example 1 using phases 1 and 2 of Table 5. Initially, the mixture became an opalescent reflective blue in color. After all of phase 2 had been added, the mixture cooled to room temperature and the pH adjusted with citric acid, a milky phase-stable emulsion was obtained. The viscosity of the emulsion was below 100 mPas. The average particle size was 169 nm.

TABLE 5

| | | | |
|---|---|---|---|
| Manufacturer | Name | INCI Name | % by wt. |

PHASE 1

| | | | |
|---|---|---|---|
| Cognis Deutschland | Lauryl glucoside | Lauryl Glucoside | 8.00 |
| GmbH &Co. KG | | | |
| Cognis Deutschland | Dehymuls ® PGPH | Dipolyhydroxystearate | 8.00 |
| GmbH &Co. KG | | | |
| Cognis Deutschland | Glycerin | Glycerin | 6.00 |
| GmbH &Co. KG | | | |
| Cognis Deutschland | Myritol ® 331 | Cocoglycerides | 4.00 |
| GmbH &Co. KG | | | |
| Cognis Deutschland | Eutanol ® G | Octyldodecanol | 2.00 |
| GmbH &Co. KG | | | |
| Cognis Deutschland | Cetiol ® AB | C12-15 Alkyl Benzoate | 4.00 |
| GmbH &Co. KG | | | |
| Cognis Deutschland | Lanette ® O | Cetearyl Alcohol | 2.00 |
| GmbH &Co. KG | | | |

PHASE 2

| | | | |
|---|---|---|---|
| Cognis Deutschland | Plantapon ® ACG 35 | Disodium Cocoyl Glutamate | 0.35 |
| GmbH &Co. KG | Preservative | Preservative | q.s. |
| | Citric Acid | Citric Acid | q.s. |
| | Water | Water | 65.51 |

pH: 5-6.5

### Example 8

Water and preservatives were added at room temperature to the nanoemulsions obtained in Examples 1-3 and 5-7. The mixture formed is particularly suitable as a sprayable lotion for cleansing wipes, particularly for the face and baby skin. A sprayable wetting solution for cleansing wipes was obtained by mixing
20% by weight of the nanoemulsion of Examples 1-3 and 5-7,
79.0% by weight water and

1.0% by weight Euxyl® K 702.

The wipes may be sprayed or impregnated. The nanoemulsion was applied in a quantity of ca. 3 g per 1 g of wipe. Wipe material: spunlace viscose 65%/polyester 35% -55 g/m².

What is claimed is:

1. A personal care article comprising:
(a) a substrate made from a substance selected from the group consisting of a paper material, a nonwoven material, and a woven material; and
(b) an ethoxylate-free nanoemulsion impregnated into the substrate, the nanoemulsion comprising:
(i) a C₈₋₁₄ alkyl oligoglycoside;
(ii) a polyol hydroxystearate;
(iii) a C₆₋₂₂ acyl glutamate;
(iv) optionally, water; and
(v) optionally, at least one auxiliary component,
wherein the particle size of the nanoemulsion is less than 1000 nm.

2. The article of claim 1 wherein (i) comprises lauryl glucoside.

3. The article of claim 1 wherein (ii) comprises polyglycerol polyhydroxystearate.

4. The article of claim 1 wherein (iii) comprises coconut oil fatty acid glutamate.

5. The article of claim 1 wherein (v) comprises sorbitan stearate.

6. The article of claim 2 wherein (ii) comprises polyglycerol polyhydroxystearate, (iii) comprises coconut oil fatty acid glutamate, and (v) comprises sorbitan stearate.

7. The article of claim 1 wherein (iii) is present in an amount of from about 1 to 50% by weight, based on the weight of the nanoemulsion.

8. The article of claim 1 wherein (iii) is present in an amount of from about 5 to 11% by weight, based on the weight of the nanoemulsion.

9. The article of claim 1 wherein (i) and (ii) are present in a ratio by weight of from about 5:1 to 1:5.

10. The article of claim 1 wherein (i) and (ii) are present in a ratio by weight of about 1:1.

11. The article of claim 1 wherein (v) is present in a ratio by weight of (v):(i)+(ii) of from about 1:1 to 1:10.

12. The article of claim 1 wherein (v) is present in a ratio by weight of (v):(i)+(ii) of from about 1:2 to 1:5.

13. The article of claim 1 further comprising at least one oil component selected from the group consisting of fatty acid esters of C₆₋₂₂ fatty acids with C₆₋₂₂ fatty alcohols, C₆₋₂₂ dialkyl carbonates, silicone oils, 1,3-dialkyl cyclohexanes and C₆₋₂₂ dialkyl ethers, said oil component being present in an amount of from about 1 to 50% by weight, based on the weight of the nanoemulsion.

14. The article of claim 1 wherein (v) comprises glycerol.

15. The article of claim 1 wherein (v) comprises a C₁₆₋₂₂ fatty alcohol, present in an amount of from about 1 to 10% by weight, based on the weight of the nanoemulsion.

16. A personal care article comprising a substrate made from a substance selected from the group consisting of a paper material, a nonwoven material and a woven material, the substrate being impregnated with an ethoxylate-free nanoemulsion containing:
(a) from about 0.5 to 5% by weight of a C₈₋₁₂ alkyl oligoglycoside;
(b) from about 0.5 to 5% by weight of a polyglycerol polyhydroxystearate;
(c) from about 0.5 to 5% by weight of a C₁₂₋₁₈ acyl glutamate;
(d) from about 0.5 to 5% by weight of a sorbitan ester of a C₆₋₂₀ fatty acid;
(e) from about 5.0 to 20% by weight of fatty acid ester of a C₆₋₂₀ fatty acid with a C₆₋₂₀ fatty alcohol;
(f) from about 1.0 to 10% by weight of a C₆₋₁₂ dialkyl carbonate;
(g) from about 0.1 to 5% by weight of a cyclomethicone;
(h) from about 1 to 10% by weight of glycerol; and
(i) optionally, water,
wherein the particle size of the nanoemulsion is less than 1000 nm.
